(19) United States
(12) Patent Application Publication
(10) Pub. No.: US 2024/0382428 A1
(43) Pub. Date: Nov. 21, 2024

(54) PROCESS FOR THE MANUFACTURE OF A SOLID PHARMACEUTICAL ADMINISTRATION FORM

(71) Applicant: Merck Patent GmbH, Darmstadt (DE)

(72) Inventors: Malte BOGDAHN, Darmstadt (DE); Stefan SCHILLER, Darmstadt (DE); Meike HARMS, Darmstadt (DE); Daniel Joseph PRICE, Darmstadt (DE); Marcel WEDEL, Darmstadt (DE); Simon GEISSLER, Darmstadt (DE); Finn BAUER, Darmstadt (DE)

(73) Assignee: Merck Patent GmbH, Darmstadt (DE)

(21) Appl. No.: 18/576,504

(22) PCT Filed: Jul. 5, 2022
(86) PCT No.: PCT/EP2022/068523

§ 371 Date: Jan. 4, 2024

(30) Foreign Application Priority Data
Jul. 8, 2021 (EP) 21184527.6

(51) Int. Cl. A61K 9/20; A61J 3/00; A61K 31/192; A61K 31/216; A61K 31/55; B33Y 10/00; B33Y 70/00; B33Y 80/00

(52) U.S. Cl. CPC A61K 9/2095; A61J 3/005; A61K 9/2009; A61K 9/2013; A61K 9/2027; A61K 9/2054; A61K 31/192; A61K 31/216; A61K 31/55; B33Y 10/00; B33Y 70/00; B33Y 80/00

(57) ABSTRACT

The present invention relates to a process for the preparation of a solid pharmaceutical administration form comprising mesoporous silica using a 3D printing process. The process is a printing process that allows the production of solid pharmaceutical administration forms comprising drug loaded mesoporous silica in an easy and flexible manner and in conformity with the high-quality standards required for the production of pharmaceutical.

The present invention relates to a process for the preparation of a solid pharmaceutical administration form comprising mesoporous silica using a 3D printing process. The process is a printing process that allows the production of solid pharmaceutical administration forms comprising drug loaded mesoporous silica in an easy and flexible manner and in conformity with the high-quality standards required for the production of pharmaceuticals.

Mesoporous silica has attracted growing interest in the development of oral drug delivery systems. Their large pore volume (up to 2.5 mL/g) in combination with a large specific surface area (up to 1,000 m²/g) results in a high drug-loading capacity. In addition, pore size and surface chemistry can be modified during synthesis to fit the application needs of the user.

Many of the recently developed active pharmaceutical ingredients (APIs) suffer from poor aqueous solubility, which leads to incomplete dissolution throughout the gastro-intestinal (GI) tract, resulting in low and variable bioavailability. A significant number of new drug candidates fail during development due to poor bioavailability, with numbers increasing. Therefore, advancement in innovative approaches to overcome this important formulation challenge is critically needed, making the development of new drug delivery systems highly desirable.

The pore structure of mesoporous silica is the key attribute to improving the dissolution rate of poorly soluble drugs. Because the pores are only a few times larger than drug molecules, the drug is confined and unable to crystallize. In this amorphous form, compounds exhibit higher dissolution rates when compared to their crystalline state, especially when the solubility is limited by high lattice energy. This in turn increases oral bioavailability, as shown by Mellaerts et al. (Eur J Pharm Biopharm 69:223-230, 2008).

Mesoporous silica loaded with API can be incorporated into solid oral dosage forms, typically tablets. Purely soluble APIs incorporated in mesoporous silica (e.g. Parteck® SLC, Merck Life Science) are stabilized in an amorphous solid state and have an increased dissolution in biorelevant media, thus improving bioavailability.

Tablets comprising API loaded mesoporous silica have several advantages as discussed above but their production typically requires many unit operations including
a) Dissolving the API in a volatile solvent
b) Impregnating the agitated mesoporous silica powder with the solution with concurrent drying and final drying
c) Blend with excipients for granulation
d) Granulation
e) Blend with excipients for compression
f) Compression

Further, pure loaded mesoporous silica powder typically exhibits poor flowability and poor compressibility/tabletability. Excipients to facilitate compression are typically added, but this increases the complexity of the formulation and decreases the maximum drug load in the final dosage form.

In the manufacture of the final dosage form, preferably a tablet, the step of loading the mesoporous silica with API remains a challenge. At lab scale, when testing the feasibility of mesoporous silica for bioavailability enhancement of a certain API, drug loading is typically done with the slurry method (mesoporous silica powder is suspended in a stirred, diluted solution of API; the solvent is slowly removed). The slurry method is good for APIs with a moderate crystallization tendency (like fenofibrate) but tends to give false negatives for APIs with a high crystallization (like carbamazepine).

While this may be overcome by using the impregnation method (diluted solution of API is sprayed onto the mesoporous silica powder), this method is not scalable downwards to lab scale and only possible if higher amounts of API are available (especially at the beginning of a development the poor availability of the API and its high costs are often a critical issue). Further challenges of this loading process is the precise control of a very slow fluid dosing rate that is necessary for loading of small amounts of mesoporous silica (too high=over-wetted silica=crystallization), and the limited ability for a reliable agitation of small amounts of mesoporous silica to evenly distribute the fluid across the mesoporous silica.

Although tablets comprising API loaded mesoporous silica offer a great opportunity to provide tablets with improved dissolution and bioavailability, such potential is difficult to be realized due to problems involved with their development and manufacture. The available process of loading of API into the silica is compromised by problems such as over-wetting of the silica with minimal droplet size of available fluid handling systems in pharmaceutical laboratories (e.g. air—cushion pipet). In addition, the poor flowability and compressibility of mesoporous silica provides further challenges to the already complex development of a conventional tablet that need to be overcome by extensive and time-consuming development work. Especially at the beginning of a development small batches are needed for testing purposes and a high degree of flexibility is needed. Thus, a simplified and flexible process that enables the fast development of solid dosage forms comprising API loaded mesoporous silica as well as their production even in small batches is highly desirable.

The present invention provides a process that meets such requirements. The invention uses an additive manufacturing process, which is also known as binder jetting. Binder Jetting is known for the manufacturing of oral solid dosage forms, the base technology was developed by MIT.

The process of the present invention is a process for the manufacture of a solid pharmaceutical administration form comprising an active ingredient comprising the steps
(a) spreading a powder comprising mesoporous silica particles across the manufacturing area to create a powder bed;
(b) jet printing a medium comprising an active pharmaceutical ingredient onto the powder;
(c) jet printing a medium comprising a binding material onto the powder;
(d) spreading a layer of powder comprising mesoporous silica particles onto the surface of powder obtained after performing step (b) and (c) and subsequently performing step (b) and step (c);
(e) repeating step (d) as often as needed to build up the solid pharmaceutical administration form;
(f) separating the solid pharmaceutical administration form from the powder bed.

In some instances it may be preferable that not each layer of powder that is spread onto the surface of powder and on which the API is jet printed in accordance to step (b) is followed by jet printing a medium comprising a binding material (step (c)) prior to spreading a new layer of powder onto the surface of powder. In such instances the sub-cycle consisting of steps (b) to (d), i.e. steps (b) jet printing a medium comprising an active pharmaceutical ingredient onto the powder, (c) jet printing a medium comprising a binding material onto the powder and (d) spreading a layer of powder comprising mesoporous silica particles onto the surface of powder obtained after performing step (b) and (c) and subsequently performing step (b) and step (c) is substituted by the sub-cycle comprising of steps (b) and (d1), i.e. (b) jet printing a medium comprising an active pharmaceutical ingredient onto the powder and step (d1) spreading a layer of powder comprising mesoporous silica particles onto the surface of powder obtained after performing step (b) and subsequently performing step (b). Such alternative sub-cycle may be run for one time, two times, three times, four times or five times in between of running the complete sub-cycle comprising steps (b), (c) and (d).

The process can be run on a 3D printer composed of a pair of horizontal X-Y axes that are suspended over a vertical piston, providing control over three directions of motion and that is equipped with jet head as known from ink jet printing technology. The printing assembly may comprise one or more jet heads that allow printing of multiple fluids successively or in parallel. A jet head may comprise multiple nozzles that may be controlled independently. A suitable jet head may work for example according to the Continuous Inkjet principle (fluid is pressurized and in a continuous stream), or the drop-on-demand principle (fluid is expelled from the jet nozzle one drop at a time). For manufacture of solid pharmaceutical dosage form powder is spread onto a mounting plate to create a powder bed, the medium is precisely distributed over predefined areas of the powder bed through a jet head that is moved over the powder bed. After lowering the mounting plate by a fixed distance, a layer of powder is spread, and the process is repeated. Instead of lowering the mounting plate the spreading means can be raised by a fixed distance.

Manufacturing of oral solid dosage forms using the process described above, wherein a binder is jet-printed to the powder bed, is known as Binder Jetting. The base technology for Binder Jetting was developed by MIT. Today's most prominent example is Aprecia's Spritam. It is believed that future improvements in disease treatment is driven by point-of-care and home-based diagnostics linked with genetic testing and emerging technologies such as proteomics and metabolomics analysis. This has led to the concept of personalized medicine, which foresees the customization of healthcare to an individual patient.

In the process of the present invention jet printing is further used for the API loading of mesoporous silica. This allows to implement the API loading as an integral step into the manufacturing process building up the solid pharmaceutical dosage form so that no separate drug loading process prior to manufacturing of the solid dosage form is necessary. Advantageously the process of the invention allows to run the API loading and dosage form manufacturing simultaneously.

Use of jet printing further improves the API loading process as it allows to decrease the minimum size of droplets comprising the API that can be precisely dosed on the mesoporous silica powder, thus reducing the amount of mesoporous silica powder needed to prevent over-wetting and crystallization. Small batch loading may be used for feasibility trials in early development phases of new APIs.

On the one hand the process of the invention omits the problem of over-wetting or agitating the material by dosing small droplets comprising API onto a thin powder surface. On the other hand, the (difficult) compression properties of mesoporous silica particles are not relevant as no plastic deformation is as in traditional tableting processes is required.

The term “solid pharmaceutical administration form” as used herein means any pharmaceutical formulation that is solid and provides a dosage unit of an active pharmaceutical ingredient that can be administered to a patient by any way of application such as oral, rectal, vaginal, implantation. The solid pharmaceutical administration form can have any shape adapted to the application requirements, e.g. round, oval, rod like, torpedo shaped etc. Examples of solid pharmaceutical administration forms are tablets, pills, caplets, suppositories, implants. Preferably, the solid pharmaceutical administration form is a tablet.

The term “active ingredient” as used herein means any ingredient that provides a pharmacological or biological effect when applied to a biological system. The active ingredient may be a pharmaceutical drug, biological matter of viral or living origin. Examples of an active ingredient that may be used in the process of the present inventions are insulin, heparin, calcitonin, hydrocortisone, prednisone, budesonide, methotrexate, mesalazine, sulfasalazine, amphotericin B, fenofibrate, carbamazepine, ibuprofen, glibenclamide, dipyridamole, itraconazole, celecoxib, haloperidol, indomethacin, posaconazole, nucleic acids, or antigens (peptides, proteins, sugars, or other substances that form surfaces recognized by the immune system, either produced, extracted, or homogenized from tissue, an organism or a virus).

The term “spreading” as used herein means a process where a planar layer of powder is applied to a planar ground. Spreading of powder can be achieved by using means that are suitable to create a planar layer of powder. Examples of such means are a doctor blade or a roller that can be moved in parallel to planar ground such as a mounting area or an existing powder layer to distribute the powder from a reservoir across the planar ground. By the use of a roller a certain level of compaction can be obtained, which may be advantageous for the manufacture of the solid pharmaceutical dosage form.

The term “mesoporous silica” refers to porous silica having medium-sized pores, specifically, pores of about 2 nanometers (nm) to about 50 nm. The term “mesoporous silica particles” refers to particulate mesoporous silica.

As used herein, “a” or “an” shall mean one or more. As used herein when used in conjunction with the word “comprising,” the words “a” or “an” mean one or more than one. As used herein “another” means at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−1-3% of the recited value) that one of ordinary skill in the art would consider equivalent to the recited value (e.g., having the same function or result). In some instances, the term “about” may include numerical values that are rounded to the nearest significant figure.

As used herein, “jet printing” refers to a process where a medium is distributed to the powder bed by ejecting droplets of medium at high speed towards and onto the powder bed. Ejection of droplets can be performed with utmost precision to predefined target place. By managing size of droplets, amount of droplets and specific target place the exact placement on and penetration depth in a substrate can be precisely controlled. Jet printing is well-known from inkjet printing technology but in contrast to this technology the medium that is printed in the process of the present invention is not an ink for printing of images but a medium, that contains materials that are usable for printing of solid pharmaceutical administration forms. In step (b) the medium comprises at least an active pharmaceutical ingredient, in step (c) the medium comprises at least a binding material.

The medium used for jet printing is a liquid or a hot melt wherein the material to be printed is distributed. The term “liquid”, as used herein, refers to solvents, that are fluid at ambient temperature (about 25° C.). Examples of liquids that can be used for distribution of the material are water, organic solvents, such as ethanol, or mixtures of both, whereby the organic solvent may be soluble with one another or not. The material may be dissolved, suspended or emulsified in the liquid. Auxiliaries such as surfactants may be used, e.g. to improve dispersibility of the material in the liquid and/or spreading or wetting of particles in the powder bed. Further examples of auxiliaries include viscosity modifiers, e.g. glycerol to enable jet printing by preventing excessive wetting of the nozzle plate, or by controlling the flow of the liquid through the channels and nozzles of the jet head; agents to control the hydrophilicity or hydrophobicity of the ink, e.g. co-solvents, such as ethanol, butanol, diethylene glycol, polyethylene glycol, dimethyl sulfoxide, hexane, to improve dispersibility of the material in the liquid and/or spreading or wetting of particles in the powder bed; humectants, e.g. glycerol or propylene glycol to prevent nozzle clogging by ink evaporation; film formers, sometimes called binders or resins, to control the spreading of the ink on the substrate, and to prevent bleeding or smearing of the ink on the substrate; dyes or pigments; and defoamers.

The term “hot melt”, as used herein, refers to a medium consisting of a material that is solid or semisolid under ambient temperature (about 25° C.) but becomes liquid (melted) at elevated temperature (operating temperature). At the jet operating temperature, droplets of melted medium comprising material other than the medium itself, e.g. an API used in step (b) or a binding material present used in step (c) is ejected from the jet head, which solidifies when coming into contact with the surface of the powder bed. Materials suitable as a medium for hot melt are, for example, polymers such as ethylene vinyl acetate (EVA), polyethylene glycol (PEG), Poly (ethylene glycol)-block-poly (propylene glycol) (PEG-PPG), poloxamers, polyvinylpyrrolidone, polyvinyl alcohol, polyvinyl acetate, a vinylpyrrolidone-vinyl acetate copolymer, a cellulose derivative, such as hydroxypropyl methylcellulose, hydroxypropyl cellulose, ethyl cellulose, hydroxypropyl methylcellulose acetate succinate or microcrystalline cellulose, a copolymer of acrylic or methacrylic acid and an acrylic or methacrylic ester, preferably a copolymer of methacrylic acid and a methacrylate or a acrylate, such as, for example Poly (methacrylic acid-co-methyl methacrylate) (1:1) (e.g. Eudragit® L 100), Poly (methacrylic acid-co-methyl methacrylate) (1:2) (e.g. Eudragit® S 100) or Poly (methacrylic acid-co-ethyl acrylate) (1:1) (e.g. Eudragit® L 100-55), a copolymer of ethyl acrylate, methyl methacrylate and a low content of methacrylic acid ester with quaternary ammonium groups, such as, for example, Poly(ethyl acrylate-co-methyl methacrylate-co-trimethylammonioethyl methacrylate chloride) 1:2:0.2 (e.g. Eudragit® RL) or Poly(ethyl acrylate-co-methyl methacrylate-co-trimethylammonioethyl methacrylate chloride) 1:2:0.1 (e.g. Eudragit® RS), a copolymer of dimethylaminoethyl methacrylate, butyl methacrylate, and methyl methacrylate, such as, for example, Poly(butyl methacrylate-co-(2-dimethylaminoethyl) methacrylate-co-methyl methacrylate) (2:1:1) (e.g. Eudragit® E PO), or waxes such as bee wax or carnauba wax. Suitable operating temperatures can be any temperature in the range from 40 to 250° C., preferably in the range from 40 to 100° C., more preferably in the range from 50 to 70° C.

If the medium used in step (b) is a hot melt it may itself act as a binding material in which case the medium used in step (c) may not need to comprise a binding material. In some instances, if a hot melt is used in step (b) that provides sufficient binding, step (c) may be omitted. Accordingly, the invention is also directed to a process for the manufacture of a solid pharmaceutical administration form, wherein the medium used in step (b) is a hot melt and step (c) is omitted.

The term “binding material”, as used herein, refers to a material that provides binding or sticking of particles. When applied onto the powder bed, the particles, that come into contact with the binding material, adhere to each other thereby generating a solid composed of particles, which are attached to each other. The binding material provides cohesion and strength to the solid preparation.

Binding materials which can be employed in the present invention are, for example, polyvinylpyrrolidone, polyvinyl alcohol, polyvinyl acetate, a vinylpyrrolidone-vinyl acetate copolymer, polyethylene glycol, a starch, such as maize starch or pre-gelatinized starch, a cellulose derivative, such as hydroxypropyl methylcellulose, hydroxypropyl cellulose, ethyl cellulose, hydroxypropyl methylcellulose acetate succinate or microcrystalline cellulose, a copolymer of acrylic or methacrylic acid and an acrylic or methacrylic ester, preferably a copolymer of methacrylic acid and a methacrylate or a acrylate, such as, for example Poly(methacrylic acid-co-methyl methacrylate) (1:1) (e.g. Eudragit® L 100), Poly(methacrylic acid-co-methyl methacrylate) (1:2) (e.g. Eudragit® S 100) or Poly(methacrylic acid-co-ethyl acrylate) (1:1) (e.g. Eudragit® L 100-55), a copolymer of ethyl acrylate, methyl methacrylate and a low content of methacrylic acid ester with quaternary ammonium groups, such as, for example, Poly(ethyl acrylate-co-methyl methacrylate-co-trimethylammonioethyl methacrylate chloride) 1:2:0.2 (e.g. Eudragit@ RL) or Poly(ethyl acrylate-co-methyl methacrylate-co-trimethylammonioethyl methacrylate chloride) 1:2:0.1 (e.g. Eudragit® RS), a copolymer of dimethylaminoethyl methacrylate, butyl methacrylate, and methyl methacrylate, such as, for example, Poly(butyl methacrylate-co-(2-dimethylaminoethyl) methacrylate-co-methyl methacrylate) (2:1:1) (e.g. Eudragit® E PO), preferably hydroxypropyl methylcellulose acetate succinate or polyvinyl alcohol, more preferably hydroxypropyl methylcellulose acetate succinate.

In principle, the process steps of process described above and below are run in a subsequent order from (a) to (f). However, this does not apply in a strict manner for the jet printing steps (b) and (c). Such printing steps cannot only be run in an order, wherein step (b) is run first which is then followed by step (c), but also in reversed order, i.e. wherein step (c) is run first which is then followed by step (b) or in a manner, wherein steps (b) and (c) are run in parallel. Accordingly, the invention is also directed to a process for the manufacture of a solid pharmaceutical administration form as set forth above, wherein steps (b) and (c) are run in parallel or subsequently in any order.

As described above the medium used in the medium jet printing usable in steps (b) and (c) can be a liquid or a hot melt. In principle, each of the steps (b) and (c), independently from each other, can use a liquid or a hot melt, whereby all combinations are possible. Accordingly, the process can be performed in a way, wherein (i) step (b) is performed using a liquid and step (c) is performed using a hot melt; (ii) step (b) is performed using a hot melt and step (c) is performed using a liquid; (iii) both of the steps (b) and (c) are performed using can use a fluid; or (iv) both of the steps (b) and (c) is performed using a hot melt. Thus, the invention is further directed to a process for the manufacture of a solid pharmaceutical administration form, wherein the medium used for jet printing in step (b) and/or step (c) is a liquid and/or a hot melt.

In the process of the present invention as described above the active pharmaceutical ingredient and the binding material are introduced into the solid pharmaceutical administration form using different printing steps, step (b) and (c). This approach allows high flexibility and a high degree of freedom in the development of the solid pharmaceutical administration form, e.g. in the selection of the media and the process parameters used for the jet printing and their adaption to the specific requirements of the materials used in the process. In some instances, however, such high flexibility and degree of freedom is not needed so that steps (b) and (c) can be combined into one step, which leads to simplification of the process and shortening of process times. Accordingly, the invention is also directed to a process for the manufacture of a solid pharmaceutical administration form, wherein steps (b) and (c) are combined into one step (step (c1)), comprising jet printing of one medium comprising the active ingredient and the binding material onto the powder. The medium usable for such process can be a fluid or a hot melt.

In an alternative embodiment of the process of the invention the binding material is added in the process not by jet-printing onto the powder but as part of the powder used in steps (a) and (d). In such embodiment the powder used in step (a) and (d) is a mixture comprising mesoporous silica particles and particles of the binding material. Further, step (c) is not necessary and can be omitted. The particular binding material does not provide binding or sticking of particles at dry state, and, thus, must be activated by a liquid, preferably by jet printing. In an advantageous embodiment, the liquid is part of the medium used in step (b) of the process.

Thus, the invention is also directed to a process for the manufacture of a solid pharmaceutical administration form comprising an active ingredient comprising the steps
(a) spreading a powder comprising mesoporous silica particles and binding material particles across the manufacturing area to create a powder bed;
(b) jet printing a liquid comprising an active pharmaceutical ingredient onto the powder;
(d) spreading a layer of powder comprising mesoporous silica particles and binding material particles onto the surface of powder obtained after performing step (b) and subsequently performing step (b);
(e) repeating step (d) as often as needed to build up the solid pharmaceutical administration form;
(f) separating the solid pharmaceutical administration form from the powder bed.

In some instances it may be difficult to provide a medium that allows to dissolve the API in the required quantities and, further, is suitable to activate binding of the binding material present in the powder, as it may be the case, for example, if dissolving of the API and activation of the binder require different polarities of the liquid. In such instances the API and the liquid for activation of the binder can be jet printed in separate steps, i.e. the API in step (b) and the liquid for activation of the binder in step (c). Obviously, as the binding material is part of the powder, no binding material needs to be present in the liquid used in step (c) of this embodiment, preferably the liquid used in step (c) does not contain a binding material. Thus, the invention is also directed to the process as described in the paragraph above, further comprising jet printing a liquid that does not comprise a binding material as step (c).

The whole process for the manufacture of a solid pharmaceutical administration form comprising an active ingredient comprises the steps
(a) spreading a powder comprising mesoporous silica particles and binding material particles across the manufacturing area to create a powder bed;
(b) jet printing a liquid comprising an active pharmaceutical ingredient onto the powder;
(c) jet printing a liquid that does not comprise a binding material;
(d) spreading a layer of powder comprising mesoporous silica particles and binding material particles onto the surface of powder obtained after performing step (b) and subsequently performing step (b);
(e) repeating step (d) as often as needed to build up the solid pharmaceutical administration form;
(f) separating the solid pharmaceutical administration form from the powder bed.

Especially if a liquid is used for jet printing steps it may be necessary to wait for some time to allow the liquid to evaporate prior to proceeding with the further steps of the process (d), (e) or (f). In such cases a drying step can be introduced to facilitate evaporation and speed up the process. Thus, the present invention is further directed to the process for the manufacture of a solid pharmaceutical administration form, wherein a drying step is performed after performing step (b) and/or step (c) or after performing step (c1).

According to a suitable embodiment the drying step is performed by using heating, lowering air pressure or convection. As each of such measures facilitates evaporation on its own, each of such measures can be combined with one or more of the other measures to achieve an additive effect and to speed up the drying step. Hence, the invention is as well directed to process for the manufacture of a solid pharmaceutical administration, wherein the drying step comprises heating, low (air) pressure, and/or convection. An appropriate low air pressure is an air pressure below atmospheric pressure, for example an air pressure in the range from 100 to 80000 Pa, preferable in the range from 5000 to 50000 Pa. Convection may be applied, for example, by an air blower. An example of an embodiment of a drying step, wherein heating is combined with convection is blowing heated air to the powder bed by use of an air blower.

Heating can be applied by infrared irradiation, a hot gas flow and/or heated surfaces. Thus, the invention is also directed to a process for the manufacture of a solid pharmaceutical administration form, wherein heating is applied by infrared irradiation, a hot gas flow and/or heated surfaces. The gas of the hot gas flow can be any gas of one element or chemical compound such as, for example, nitrogen or carbon dioxide, or a mixture of elements and chemical compounds gases, such as air. Heated surfaces can be provided, for example, by heating the mounting plate or heating part or all of the encasement of the 3D printer used for running the process.

In principle, a solid pharmaceutical administration form for any way of application such as oral, rectal, vaginal, implantation can be manufactured with the process as described herein. However, the process is especially suitable for the manufacture of a solid pharmaceutical administration form for oral use. Thus, an advantageous embodiment of the invention described herein is directed to a process for manufacture of a solid pharmaceutical administration form, wherein the pharmaceutical administration form is for oral use.

As explained above API loading in mesoporous silica leads to stabilization of the API in an amorphous solid state and to an increased dissolution in biorelevant media. Thus, the process of the invention is especially suitable for manufacture of immediate release formulations. Accordingly, a preferred embodiment of the invention is directed to the process for manufacture of a solid pharmaceutical administration form, wherein the pharmaceutical administration form provides immediate release of the active pharmaceutical ingredient.

The term “immediate release”, as used herein, means that the majority of the active pharmaceutical ingredient is released quickly from the pharmaceutical administration form. Preferably, at least 80 percent of the active is released within 30 minutes from administration, more preferably within 15 minutes. The release of the API from the pharmaceutical administration form (the dissolution) is measured in pH 6.8 buffer or 0.1 N HCl using conventional dissolution testing in line with standard dissolution tests described in applicable Pharmacopoeias (e.g. Chapter 711 USP).

According to a suitable embodiment the mesoporous silica used in the process of the invention has a mean diameter from about 1 μm to about 500 μm, preferably from about 1 μm to 100 μm and more preferably from about 10 μm to 50 μm. Therefore, invention is also directed to the process for manufacture of a solid pharmaceutical administration, wherein the mesoporous silica particles have a mean diameter from about 1 μm to about 500 μm, preferably from about 1 μm to 100 μm and more preferably from about 10 μm to 50 μm.

The term “median diameter” as used herein refers to a median volume diameter (d₅₀) which can be measured by laser diffraction/scattering method. The d₅₀ value referred to herein is the size in micrometres that splits the distribution with half above and half below this diameter. The d₅₀ is the median for a volume distribution and is often also designated Dv50 (or Dv0.5).

The median diameter as disclosed herein is measured by dynamic image analysis on a Microtrac Camsizer X2 (dry dispersion method, gap width 4 mm, dispersive air pressure of 50-100 kPa, automatic feeding, nominal covered area 0.3% (limit CCD-Basic<1%; CCD-Zoom<2%).

Preferably, the mesoporous silica used in the process of the invention further has a particle size of the mesoporous silica particles is characterized by a doo value of 500 μm or less, preferably 100 μm or less and more preferably 60 μm or less, for instance between 40 μm and 60 μm. Thus, the invention is also directed to the process for manufacture of a solid pharmaceutical administration, wherein the particle size of the mesoporous silica particles is characterized by a d₉₀ value of 500 μm or less, preferably 100 μm or less and more preferably 60 μm or less, for instance between 40 μm and 60 μm.

The d₉₀ values referred to herein refer the point in the volume size distribution (size in micrometres), up to and including which, 90% of the total volume of material in the sample is ‘contained’. The method for measuring the size distribution is the same as stated above (dynamic image analysis on a Microtrac Camsizer X2). The d₉₀ values are often also designated Dv90 (or Dv0.9).

According to a suitable embodiment the mesoporous silica used in the process has an arithmetic mean pore size diameter from 2 to 50 nm. Therefore, the invention is further directed to the process for manufacture of a solid pharmaceutical administration form, wherein the mesopores of the mesoporous silica particles have a mean diameter from 2 to 50 nm.

The pore size of mesopores and their specific surface area can be determined using nitrogen adsorption/desorption measurements (BET-method), e.g. by using Accelerated Surface Area and Porosimetry System ASAP® 2420 from Micromeritics Instrument Corporation, 4356 Communications Drive, Norcross, GA 30093-2901, USA.

In some instances, when using a fluid for in one or more jet printing steps, the pharmaceutical administration form, after it was built up by execution of steps (a) to (e) and separated from the powder bed (step (f)), may still contain some residual fluid not evaporated so far. In such cases it could be necessary to remove the fluid with a subsequent drying step. Thus, the invention is also directed to the process for manufacture of a solid pharmaceutical administration form, wherein a drying step is performed after step (f). Such drying step comprises heating, low (air) pressure, and/or convection.

The process for manufacture of a solid pharmaceutical administration form, wherein the drying step is performed in the presence of an entrainer.

In some instances, the solubility characteristics of an API or a binder may require the use of less volatile media. Such media may be very slow or even inadequate to remove by conventional drying alone. One possibility to facilitate the removal of less volatile media may be to introduce a further solvent as entrainer. An entrainer may act as a carrier for a less volatile medium during drying, for example by forming an azeotrope with the less volatile medium. An entrainer may also increase the diffusivity of a solvent out of the solid particles, for example by increasing the molecular mobility of fully or partially amorphous solids. Suitable ways to introduce an entrainer would be for example by impregnation, annealing with a vapor containing the entrainer, or to include the entrainer into the stream of drying gas in a tray dryer or agitated vacuum dryer.

### EXAMPLES

### Printing Apparatus

The following examples were manufactured with an apparatus comprising a powder bed which can be moved in x and y direction of the machine and serves as manufacturing area for the described objects. A print head assembly is located over the powder bed. The assembly comprises one or two modified HP C6602 ink jet cartridges. The cartridges are connected to an electronic circuit which can activate the nozzles to eject droplets of fluids synchronized to the motions of the powder bed. The cartridges are modified in a way that the ink contained in the stock cartridges can the replaced. Furthermore, a connector is introduced to connect the cartridges to a pressure regulator. A negative pressure of 20 mm H₂O is applied to the ink reservoir of the cartridges. For jet printing an API containing medium the effective nozzle pitch was adjusted while for jet printing a medium comprising a binder the native nozzle pitch of the cartridge was used. At a different position a powder reservoir is mounted over the build plate comprising a powder. The material can be deposited onto the manufacturing area of the powder bed in a controlled manner.

The printing process is controlled via software commands executed in a sequential order. First a thin powder layer is prepared in the powder bed. Afterwards the powder bed is moved under the printing assembly and a liquid material is jet printed onto the surface of the powder bed. After all printing commands and optional dwell times for a specific layer are executed the next layer of particulate material (height: 0.1 mm) is deposited onto the already prepared powder bed surface and liquid material is jet printed onto the new powder layer. The process is repeated until all layers of the objects were printed.

The printing pattern as well as the necessary motions to manufacture a specific object are defined by a software which takes a digital 3-dimensional model and a settings file.

The shape of printed objects can be defined via a digital 3 dimensional model.

Each nozzle of the print head ejected 500 droplets per second maximum.

### Droplet Volume

The Droplet volume of the print heads is measured by printing a defined number of droplets in the cavities of an acrylic 96-well plate. The deposited material is diluted, and concentration of the incorporated dye is determined via UV/VIS spectroscopy. The droplet volume is calculated with the following formula:

$${V}_{droplet} = \frac{{c}_{{dye}_{sample} \times {V}_{sample}}}{{c}_{dye \_ ink} \times {n}_{droplets}}$$

### Glossary

PVA: Polyvinyl alcohol (Ph.Eur. 10.5 monograph “Poly(vinyl alcohol))
HPMC AS: Hydroxypropyl methylcellulose acetate succinate (USP-NF monograph “Hypromellose Acetate Succinate”, DocID: 1_GUID-B85C7C87-E41C-465D-8A42-E40650FE1949_6_en-US)
Kollidon VA64: copolymer of 1-vinyl-2-pyrrolidone and vinyl acetate in a ratio of 6:4 by mass (Ph Eur. 10.7 monograph “Copovidone”)
Kollidon K25: poly(1-vinyl-2-pyrrolidone) (Ph Eur. 10.7 monograph “Povidone”)

### Dissolution

1.2 ml of FaSSIF was prepared from FaSSIF/FeSSIF/FaSSGF powder (Biorelevant.com Ltd, London, United Kingdom) (L. Klumpp, Dissolution behavior of various drugs in different FaSSIF versions, European Journal of Pharmaceutical Sciences, 2020) according to instructions and heated to 37° C. in Eppendorf caps. After introducing the weighed sample into the medium the Eppendorf cap was vortexed. Before a sample was drawn, the medium was centrifuged and 50 μl of the supernatant was sampled. After sampling the solid fraction was re-suspended by vortexing. The sample was diluted with organic solvent to prevent precipitation of the API and concentration of the API in the samples was determined via HPLC.

The drug load of the objects was determined by diluting the medium after the dissolution experiment with organic solvent to solve the API comprehensively and concentration of the API was determined via HPLC. The mass of API in the printed object was calculated from all drawn samples and the end value.

### Example A

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving PVA in demineralized water to a concentration of 5.215% (m/m). The concentration of dye (methylene blue) in medium comprising a binder is 0.304 mg/ml. A powder bed comprising mesoporous silica particles is prepared. The API containing medium is jet printed onto the surface of the powder bed with 100 droplets per mm in printing direction with an effective nozzle pitch of 0.088 mm perpendicular to the printing direction. The average droplet volume is 110 pl. The medium containing a binder is jet printed onto the surface of the powder bed with 250 droplets per mm in printing direction (average droplet volume: 138 pl). After jet printing the medium containing a binder the process was interrupted for 10 seconds to let the ink solvents evaporate. A new layer of powder (0.1 mm) is applied onto the surface of the powder bed and the printing process is repeated for 20 layers in total.

The described process results in objects (shape: cylinder with diameter=6 mm and height=2 mm) with a mass of 34.97 mg±6.13 mg (mean±sd, n=6) and drug load of 1.07%±0.09% (mean±sd, n=6).

### Example B

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving PVA in demineralized water to a concentration of 5.215% (m/m). The concentration of dye (methylene blue) in medium comprising a binder is 0.304 mg/ml. A powder bed comprising mesoporous silica particles is prepared. The API containing medium is jet printed onto the surface of the powder bed with 100 droplets per mm in printing direction with an effective nozzle pitch of 0.088 mm perpendicular to the printing direction. The medium containing a binder is jet printed onto the surface of the powderbed with 300 droplets per mm in printing direction. After jet printing the medium containing a binder the powder bed is irradiated via a halogen lamp for 60 seconds with a power of 424 W to facilitate the evaporation of ink solvents. A new layer of powder (0.1 mm) is applied onto the surface of the powder bed and the printing process is repeated for 20 layers in total. The described process results in objects (shape: cylinder with diameter=6 mm and height=2 mm) with a mass of 39.35 mg±2.27 mg (mean±sd, n=3) and drug load of 1.06%±0.02% (mean±sd, n=3).

### Example C

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving PVA in demineralized water to a concentration of 5.215% (m/m). The concentration of dye (methylene blue) in medium comprising a binder is 0.304 mg/ml. A powder bed comprising mesoporous silica particles is prepared. The API containing medium is jet printed onto the surface of the powder bed with 100 droplets per mm in printing direction with an effective nozzle pitch of 0.088 mm perpendicular to the printing direction. The medium containing a binder is jet printed onto the surface of the powderbed with 500 droplets per mm in printing direction. After jet printing the medium containing a binder the powder bed is irradiated via a halogen lamp for 180 seconds with a power of 424 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 42.66 mg±1.97 mg (mean±sd, n=6) and drug load of 0.75%±0.03% (mean±sd, n=6).

### Example D

The medium comprising a binder is prepared by solving HPMC AS in methanol to a concentration of 24.7383 mg/ml solvent. The concentration of dye (methylene blue) in medium comprising a binder was 0.3017 mg/ml solvent. A powder bed of Ibuprofen-preloaded mesoporous silica particles is prepared. For loading the mesoporous silica the material is transferred into an agitated dryer. The API is dissolved in an organic solvent and API solution is slowly added to the powder in a controlled manner. After the loading process a drying step is used to remove residual solvent. The final product is loaded with 29.5% (m/m) API. Afterwards a powder bed comprising Ibuprofen loaded mesoporous silica powder is prepared and the medium containing a binder is jet printed onto the surface of the powder bed with 500 droplets per mm in printing direction. A new layer of powder (0.1 mm) is applied onto the surface of the powder bed and the printing process is repeated for 20 layers in total.

The described process results in objects (shape: cylinder with diameter=6 mm and height=2 mm) with a mass of 44.74 mg±0.50 mg (mean±sd, n=3) and drug load of 28.72%±0.39% (mean±sd, n=3).

### Example E

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving HPMC AS in methanol to a concentration of 24.7383 mg/ml solvent. The concentration of dye (methylene blue) in medium comprising a binder is 0.3017 mg/ml solvent. A powder bed comprising mesoporous silica particles is prepared. The API containing medium is jet printed onto the surface of the powder bed with 200 droplets per mm in printing direction with an effective nozzle pitch of 0.088 mm perpendicular to the printing direction. The medium containing a binder is jet printed onto the surface of the powder bed with 400 droplets per mm in printing direction. After jet printing the medium containing a binder the powder bed is irradiated via a halogen lamp for 30 seconds with a power of 424 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 33.60 mg±2.71 mg (mean±sd, n=3) and drug load of 1.99%±0.11% (mean±sd, n=3). The tensile strength of the manufactured objects is 0.99 MPa±0.15 MPa (mean±sd, n=3).

### Example F

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving HPMC AS in methanol to a concentration of 24.7383 mg/ml solvent. The concentration of dye (methylene blue) in medium comprising a binder is 0.3017 mg/ml solvent. A powder bed comprising mesoporous silica particles is prepared. The API containing medium is jet printed onto the surface of the powder bed with 200 droplets per mm in printing direction with an effective nozzle pitch of 0.088 mm perpendicular to the printing direction. The medium containing a binder is jet printed onto the surface of the powder bed with 500 droplets per mm in printing direction. After jet printing the medium containing a binder the powder bed is irradiated via a halogen lamp for 60 seconds with a power of 424 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 38.03 mg±1.71 mg (mean±sd, n =3) and drug load of 2.06%±0.08% (mean±sd, n=3). The tensile strength of the manufactured objects is 1.54 MPa±0.14 MPa (mean±sd, n=3).

### Example G

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving HPMC AS in methanol to a concentration of 24.7383 mg/ml solvent. The concentration of dye (methylene blue) in medium comprising a binder is 0.3017 mg/ml solvent. A powder bed comprising mesoporous silica particles is prepared. The API containing medium is jet printed onto the surface of the powder bed with 200 droplets per mm in printing direction with an effective nozzle pitch of 0.088 mm perpendicular to the printing direction. The medium containing a binder is jet printed onto the surface of the powder bed with 500 droplets per mm in printing direction. After jet printing the medium containing a binder the powder bed is irradiated via a halogen lamp for 60 seconds with a power of 424 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 38.03 mg±1.71 mg (mean±sd, n=3) and drug load of 2.16%±0.11% (mean±sd, n=3). The tensile strength of the manufactured objects is 1.49 MPa±0.14 MPa (mean±sd, n=3).

### Example H

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving HPMC AS in methanol to a concentration of 24.7383 mg/ml solvent. The concentration of dye (methylene blue) in medium comprising a binder is 0.3017 mg/ml solvent. A powder bed comprising mesoporous silica particles is prepared. The API containing medium is jet printed onto the surface of the powder bed with 200 droplets per mm in printing direction with an effective nozzle pitch of 0.088 mm perpendicular to the printing direction. The medium containing a binder is jet printed onto the surface of the powderbed with 500 droplets per mm in printing direction. After jet printing the medium containing a binder the powder bed is irradiated via a halogen lamp for 60 seconds with a power of 459 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 39.45 mg±0.44 mg (mean±sd, n =3) and drugload of 2.06%±0.04% (mean±sd, n=3). The tensile strength of the manufactured objects is 1.59 MPa±0.11 MPa (mean±sd, n=3).

### Example I

To prepare the API containing medium Ibuprofen is dissolved in ethanol with a concentration of 20.0 mg/ml. A dye (Eosin Y) is added in a concentration of 0.25 mg/ml. The medium comprising a binder is prepared by solving HPMC AS in methanol to a concentration of 24.7383 mg/ml solvent. The concentration of dye (methylene blue) in medium comprising a binder is 0.3017 mg/ml solvent. A powder bed comprising mesoporous silica particles is prepared. The API containing medium is jet printed onto the surface of the powder bed with 100 droplets per mm in printing direction with an effective nozzle pitch of 0.088 mm perpendicular to the printing direction. The medium containing a binder is jet printed onto the surface of the powderbed with 500 droplets per mm in printing direction. After jet printing the medium containing a binder the process was interrupted for 60 seconds to let the ink solvents evaporate. A new layer of powder (0.1 mm) is applied onto the surface of the powder bed and the printing process is repeated for 20 layers in total.

The described process results in objects (shape: cylinder with diameter=6 mm and height=2 mm) with a mass of 41.57 mg±1.85 mg (mean±sd, n=3) and drugload of 1.14%±0.18% (mean±sd, n=3). The tensile strength of the manufactured objects is 0.69 MPa±0.09 MPa (mean±sd, n=3).

### Example J

To prepare the API containing medium Ibuprofen is dissolved in ethanol with a concentration of 20.0 mg/ml. A dye (Eosin Y) is added in a concentration of 0.25 mg/ml. The medium comprising a binder is prepared by solving HPMC AS in methanol to a concentration of 24.7383 mg/ml solvent. The concentration of dye (methylene blue) in medium comprising a binder is 0.3017 mg/ml solvent. A powder bed comprising mesoporous silica particles is prepared. The API containing medium is jet printed onto the surface of the powder bed with 200 droplets per mm in printing direction with an effective nozzle pitch of 0.088 mm perpendicular to the printing direction. The medium containing a binder is jet printed onto the surface of the powderbed with 500 droplets per mm in printing direction. After jet printing the medium containing a binder the process was interrupted for 120 seconds to let the ink solvents evaporate. A new layer of powder (0.1 mm) is applied onto the surface of the powder bed and the printing process is repeated for 20 layers in total.

The described process results in objects (shape: cylinder with diameter=6 mm and height=2 mm) with a mass of 39.72 mg±1.29 mg (mean±sd, n=3) and drugload of 1.70%±0.06% (mean±sd, n=3). The tensile strength of the manufactured objects is 0.80 MPa±0.05 MPa (mean±sd, n=3).

### Example K

To prepare the API containing medium Fenofibrate is dissolved in ethanol with a concentration of 19.83 mg/ml. A dye (Eosin Y) is added in a concentration of 0.25 mg/ml. The medium comprising a binder is prepared by solving PVA in demineralized water to a concentration of 5.215% (m/m). The concentration of dye (methylene blue) in medium comprising a binder is 0.304 mg/ml. A powder bed comprising mesoporous silica particles is prepared. The API containing medium is jet printed onto the surface of the powder bed with 600 droplets per mm in printing direction with an effective nozzle pitch of 0.265 mm perpendicular to the printing direction. The medium containing a binder is jet printed onto the surface of the powderbed with 150 droplets per mm in printing direction. A new layer of powder (0.1 mm) is applied onto the surface of the powder bed and the printing process is repeated for 20 layers in total.

The described process results in objects (shape: rectangle (10 mm×9.5 mm)) with a mass of 20.55 mg±2.22 mg (mean±sd, n=3) and drugload of 4.21%±0.57% (mean±sd, n=3).

### Example L

To prepare the API containing medium Carbamazepine is dissolved in methanol with a concentration of 40.1533 mg/ml. A dye (Eosin Y) is added in a concentration of 0.25 mg/ml. The medium comprising a binder is prepared by solving HPMC AS in methanol to a concentration of 24.7383 mg/ml solvent. The concentration of dye (methylene blue) in medium comprising a binder is 0.3017 mg/ml solvent. A powder bed comprising mesoporous silica particles is prepared. The API containing medium is jet printed onto the surface of the powder bed with 200 droplets per mm in printing direction with an effective nozzle pitch of 0.088 mm perpendicular to the printing direction. The medium containing a binder is jet printed onto the surface of the powderbed with 500 droplets per mm in printing direction. After jet printing the medium containing a binder the powder bed is irradiated via a halogen lamp for 60 seconds with a power of 424 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 46.34 mg±0.36 mg (mean±sd, n=3) and drugload of 5.95%±0.10% (mean±sd, n=3). The tensile strength of the manufactured objects is 1.22 MPa±0.11 MPa (mean±sd, n=3).

### Example M

To prepare the API containing medium Fenofibrate was dissolved in Ethanol with a concentration of 19.98 mg/ml. A Dye (Eosin Y) was added in a concentration of 0.25 mg/ml. The medium comprising a binder was prepared by solving HPMC AS in methanol to a concentration of 24.16 mg/ml. The concentration of dye (methylene blue) in medium comprising a binder was 0.29 mg/ml. A powder bed comprising mesoporous silica particles is prepared. The API containing medium was jet printed onto the surface of the powder bed with 600 droplets per mm in printing direction with an effective nozzle pitch of 0.265 mm perpendicular to the printing direction. After jet printing the medium containing the API the powder bed is irradiated via a halogen lamp for 30 seconds with a power of 424 W to facilitate the evaporation of ink solvent. Jet printing of the medium containing a binder and irradiation of powderbed was repeated for 5 times in total. The medium containing a binder is jet printed onto the surface of the powderbed with 1000 droplets per mm in printing direction. After jet printing the medium containing a binder the powder bed is irradiated via a halogen lamp for 30 seconds with a power of 424 W to facilitate the evaporation of ink solvent. A new layer of powder (0.1 mm) is applied onto the surface of the powder bed and the printing process is repeated for 4 layers in total.

The described process results in objects (shape: rectangle with dimensions of 5 mm×3.1 mm) with a mass of 3.08 mg±0.02 mg (mean±sd, n=3) and drug load of 26.76%±2.83% (mean±sd, n=3).

### Example N

To prepare the API containing medium Fenofibrate was dissolved in Ethanol with a concentration of 19.98 mg/ml. A Dye (Eosin Y) was added in a concentration of 0.25 mg/ml. The medium comprising a binder was prepared by solving Kollidon K25 in ethanol to a concentration of 42.84 mg/ml. The concentration of dye (methylene blue) in medium comprising a binder was 0.29 mg/ml. A powder bed comprising mesoporous silica particles is prepared. The API containing medium was jet printed onto the surface of the powder bed with 200 droplets per mm in printing direction with an effective nozzle pitch of 0.088 mm perpendicular to the printing direction. The medium containing a binder is jet printed onto the surface of the powderbed with 1250 droplets per mm in printing direction. After jet printing the medium containing a binder the powder bed is irradiated via a halogen lamp for 60 seconds with a power of 424 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 41.44 mg and 41.62 mg and drug load of 1.84% and 1.82%.

### Example O

To prepare the API containing medium Fenofibrate was dissolved in Ethanol with a concentration of 19.98 mg/ml. A Dye (Eosin Y) was added in a concentration of 0.25 mg/ml. The medium comprising a binder was prepared by solving Kollidon VA64 in ethanol to a concentration of 40.10 mg/ml. The concentration of dye (methylene blue) in medium comprising a binder was 0.15 mg/ml. A powder bed comprising mesoporous silica particles is prepared. The API containing medium was jet printed onto the surface of the powder bed with 200 droplets per mm in printing direction with an effective nozzle pitch of 0.088 mm perpendicular to the printing direction. The medium containing a binder is jet printed onto the surface of the powderbed with 1250 droplets per mm in printing direction. After jet printing the medium containing a binder the powder bed is irradiated via a halogen lamp for 60 seconds with a power of 424 W to facilitate the evaporation of ink solvents. A new layer of powder (0.1 mm) is applied onto the surface of the powder bed and the printing process is repeated for 20 layers in total.

The described process results in objects (shape: cylinder with diameter=6 mm and height=2 mm) with a mass of 40.46 mg and 35.51 mg and drug load of 1.84% and 2.07%.

### Example P

To prepare the API containing medium Fenofibrate was dissolved in ethanol with a concentration of 20.12 mg/ml. A Dye (Eosin Y) was added in a concentration of 0.25 mg/ml. A powder bed comprising mesoporous silica particles and 25% (m/m) Kollidon VA64 particles is prepared. The API containing medium was jet printed onto the surface of the powder bed with 200 droplets per mm in printing direction with an effective nozzle pitch of 0.132 mm perpendicular to the printing direction. After jet printing the medium containing an API the process was interrupted for 60 seconds to let the ink solvent evaporate. A new layer of powder (0.1 mm) is applied onto the surface of the powder bed and the printing process is repeated for 20 layers in total.

The described process results in objects (shape: cylinder with diameter=6 mm and height=2 mm) with a mass of 26.03 mg±0.20 mg (mean±sd, n=3) and drug load of 3.11%±0.01% (mean±sd, n=3).

### Example Q

To prepare a medium containing the API and comprising a binder Fenofibrate was dissolved in methanol with a concentration of 5.70 mg/ml and HPMC AS was dissolved to a concentration of 17.81 mg/ml in the same medium. The concentration of dye (methylene blue) in medium comprising the API and a binder was 0.21 mg/ml. A powder bed comprising mesoporous silica particles is prepared. The medium containing the API and a binder was jet printed onto the surface of the powder bed with 700 droplets per mm in printing direction with an effective nozzle pitch of 0.132 mm perpendicular to the printing direction. After jet printing the medium containing the API and a binder the powder bed is irradiated via a halogen lamp for 60 seconds with a power of 424 W to facilitate the evaporation of ink solvents. A new layer of powder (0.1 mm) is applied onto the surface of the powder bed and the printing process is repeated for 20 layers in total.

The described process results in objects (shape: rectangle with dimensions of 5 mm×3.1 mm) with a mass of 47.79 mg±1.17 mg (mean±sd, n=3) and drug load of 3.23%±0.06% (mean±sd, n=3).

### Example R

Fenofibrate was triturated with 50% (m/m) mannitol. Samples with a mass of 1.67 mg±0.01 mg (mean±sd, n=6) were introduced into the described dissolution setup.

The invention is illustrated in the Figures.

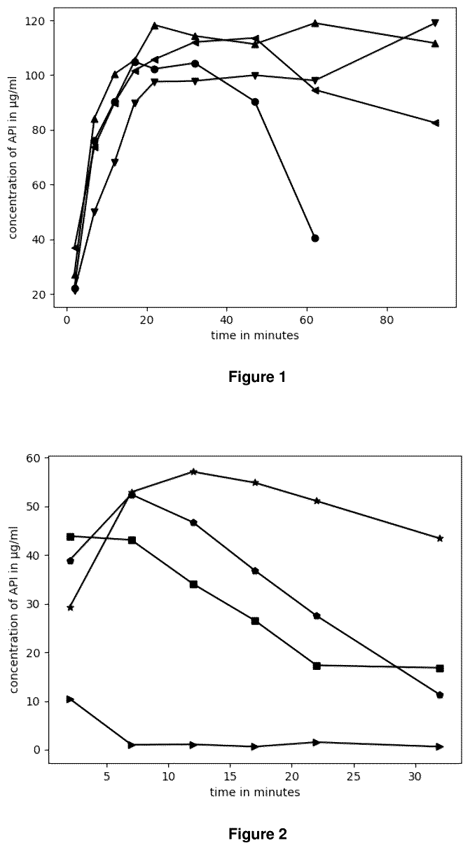

### FIG. 1

FIG. 1 shows dissolution curves of formulation prototypes (circle: Example E (mean, n=3); triangle down: Example F (mean, n=3); triangle up: Example G (mean, n=3); triangle left: Example H (mean, n=3))

All Examples showed even higher supersaturation of API in dissolution medium than Example A-C. In contrast to Examples A-C no additional precipitation inhibitor was necessary to stabilize supersaturation in these experiments.

Example E was printed with less binder than the other shown examples. These printing parameters resulted in less stable supersaturation of the API in dissolution medium, indicating, that the used binder material acts additionally as a precipitation inhibitor.

### FIG. 2

FIG. 2 shows dissolution curves of formulation prototypes (triangle right: Example A (mean, n=3); square: Example A (mean, n=3) (precipitation inhibitor was added to dissolution medium); pentagon: Example B (mean, n=3) (precipitation inhibitor was added to dissolution medium); star: Example C (mean, n=6) (precipitation inhibitor was added to dissolution medium)

Precipitation inhibitor (HPMC AS) was added to the dissolution medium in a concentration of 0.1% (m/m).

Example A without addition of precipitation inhibitor showed no supersaturation of API in dissolution medium. Sample with additional precipitation inhibitor showed supersaturation.

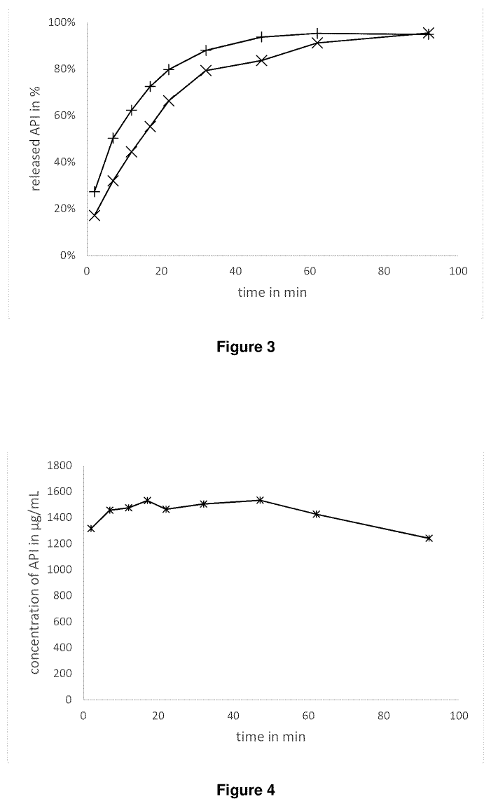

### FIG. 3

FIG. 3 shows dissolution curves of formulation prototypes (cross: Example I (mean, n=3); x: Example J (mean, n=3))

Example I: This formulation released 80% of API in less than 30 min meeting the criteria for immediate release solid oral dosage forms.

### FIG. 4

FIG. 4 shows dissolution curves of formulation prototypes (star: Example D (mean, n=3))

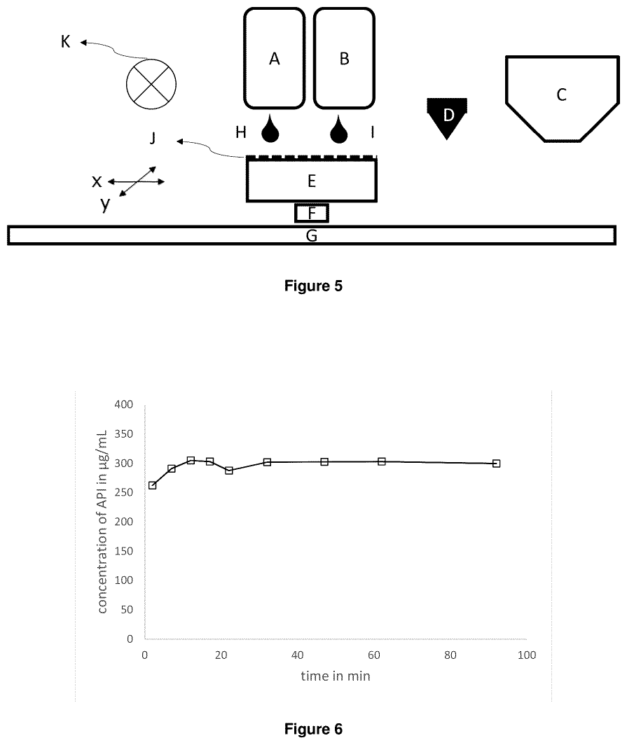

### FIG. 5

FIG. 5 shows a scheme of the printing apparatus. The build plate (E) is control system. By means of the axes the build plate can be moved to different locations aligning it with additional parts of the printing apparatus. For the manufacturing of a single layer the build plate is moved to the powder supply (C) where powder is applied to the surface of the build plate or the surface of the powder bed. A blade (D) is used to spread a thin layer of powder (J) while the build plate is moved under the blade in a linar motion. Jet printing assemblies (A+B) can be used to apply droplets of fluids (H+I) onto the surface of the powder layer in a spacially controlled manner. A halogen lamp (K) can be used to irradiate the powder bed.

### FIG. 6

FIG. 6 shows dissolution curves of formulation prototypes (hollow square: Example L (mean, n=3))

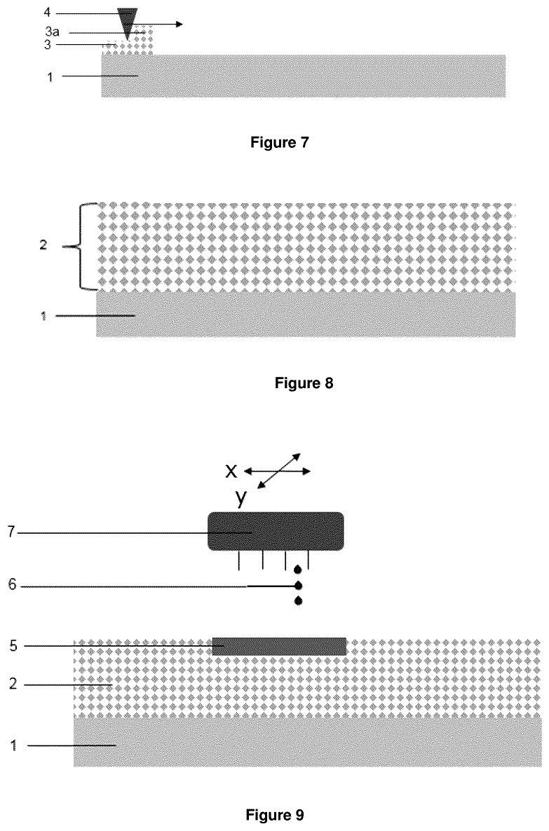

### FIG. 7

FIG. 7 illustrate the spreading step (a) of the process. Onto a mounting plate (1) a powder provided by a powder reservoir (3a) is spread by moving a doctor blade (4) in the direction indicated by an arrow to achieve a powder layer. A part of the powder layer that is already spread is indicated by (3). By repeating of the spreading of powder on the already existing powder layer(s) as often as necessary a powder bed (2) is created.

### FIG. 8

FIG. 8 shows the powder bed (2) that is created by step (a) on the mounting plate.

### FIG. 9

FIG. 9 shows jet printing in accordance to step (b) or (c) of the process. A jet head (7) is moved along x and/or y axis thereby jet printing a fluid (6) (in fine droplets) onto the powder bed (2). Such jet printing results in powder soaked with fluid (5) created by voxels that are adjacent to one another.

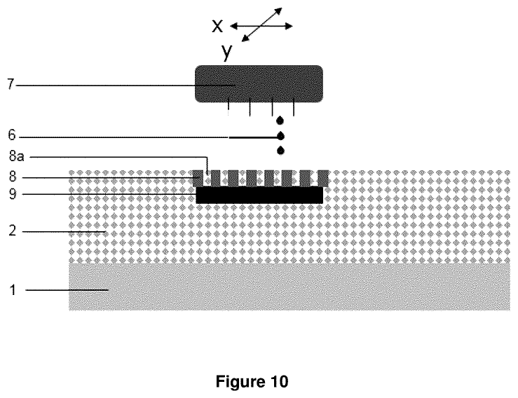

### FIG. 10

FIG. 10 shows the jet printing step as in FIG. 9 whereby the intermediate product shown in FIG. 9, onto which a layer of powder was spread, is used. In contrast to FIG. 9 the fluid is not jet printed on a continuous area but on defined areas of the powder that are delimited from each other so that a layer of powder voxels soaked with fluid (8) and powder voxels without fluid (8a) are created.

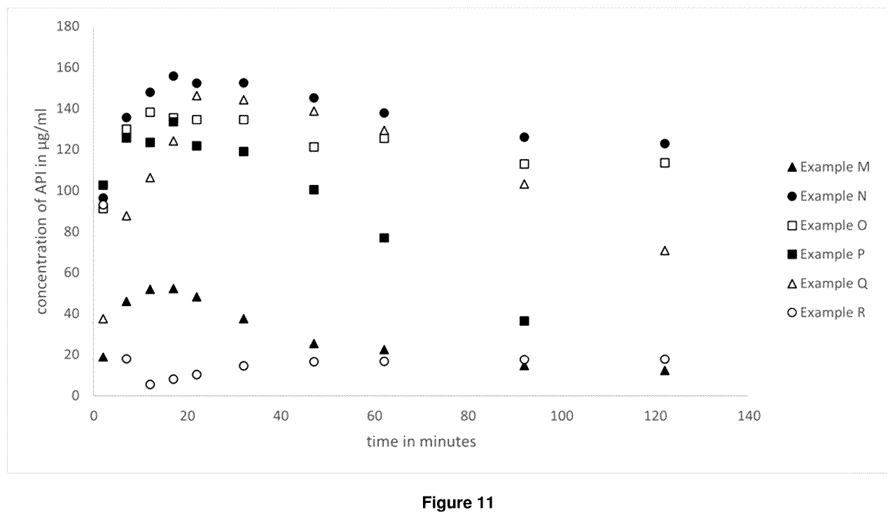

### FIG. 11

FIG. 11 shows dissolution curves of formulation prototypes (triangle solid: Example M (mean, n=3); circle solid: Example N (mean, n=2) (precipitation inhibitor was added to dissolution medium); square open: Example O (mean, n=2) (precipitation inhibitor was added to dissolution medium); square solid: Example P (mean, n=3) (precipitation inhibitor was added to dissolution medium); triangle open: Example Q (mean, n=3); circle open: Example R (mean, n=6))

Precipitation inhibitor (HPMC AS) was added to the dissolution medium in a concentration of 0.1% (m/m).

All samples except the neat fenofibrate samples showed supersaturation. Samples with binder incorporated in a jet printed medium showed supersaturation. Samples with binder incorporated into powderbed showed supersaturation. Samples with higher drugload (Example M) showed supersaturation.

1. A process for the manufacture of a solid pharmaceutical administration form comprising an active ingredient comprising the steps
(a) spreading a powder comprising mesoporous silica particles across the manufacturing area to create a powder bed;
(b) jet printing a medium comprising an active pharmaceutical ingredient onto the powder;
(c) jet printing a medium comprising a binding material onto the powder;
(d) spreading a layer of powder comprising mesoporous silica particles onto the surface of powder obtained after performing step (b) and (c) and subsequently performing step (b) and step (c);
(e) repeating step (d) as often as needed to build up the solid pharmaceutical administration form;
(f) separating the solid pharmaceutical administration form from the powder bed.

2. The process for the manufacture of a solid pharmaceutical administration form according to claim 1, wherein in steps (b) and (c) are run in parallel or subsequently in any order.

3. The process for the manufacture of a solid pharmaceutical administration form according to claim 1, wherein the medium used for jet printing in step (b) and/or step (c) is a fluid and/or a hot melt.

4. The process for the manufacture of a solid pharmaceutical administration form according to claim 1, wherein the medium used in step (b) is a hot melt and step (c) is omitted.

5. The process for the manufacture of a solid pharmaceutical administration form according to claim 1, wherein steps (b) and (c) are combined into one step (step (c1)), comprising jet printing of one medium comprising the active ingredient and the binding material onto the powder.

6. A process for the manufacture of a solid pharmaceutical administration form comprising an active ingredient comprising the steps
(a) spreading a powder comprising mesoporous silica particles and binding material particles across the manufacturing area to create a powder bed;
(b) jet printing a liquid comprising an active pharmaceutical ingredient onto the powder;
(d) spreading a layer of powder comprising mesoporous silica particles and binding material particles onto the surface of powder obtained after performing step (b) and subsequently performing step (b);
(e) repeating step (d) as often as needed to build up the solid pharmaceutical administration form;
(f) separating the solid pharmaceutical administration form from the powder bed.

7. A process for the manufacture of a solid pharmaceutical administration form comprising an active ingredient comprises the steps
(a) spreading a powder comprising mesoporous silica particles and binding material particles across the manufacturing area to create a powder bed;
(b) jet printing a liquid comprising an active pharmaceutical ingredient onto the powder;
(c) jet printing a liquid that activates binding of the powder;
(d) spreading a layer of powder comprising mesoporous silica particles and binding material particles onto the surface of powder obtained after performing step (b) and subsequently performing step (b);
(e) repeating step (d) as often as needed to build up the solid pharmaceutical administration form;
(f) separating the solid pharmaceutical administration form from the powder bed.

8. The process for the manufacture of a solid pharmaceutical administration form according to claim 1, wherein a drying step is performed after performing step (b) and/or step (c) or after performing step (c1).

9. The process for the manufacture of a solid pharmaceutical administration form according to claim 8, wherein the drying step comprises heating, low (air) pressure, and/or convection.

10. The process for the manufacture of a solid pharmaceutical administration form according to claim 9, wherein heating is applied by infrared irradiation, a hot gas flow and/or heated surfaces.

11. The process for manufacture of a solid pharmaceutical administration form according to claim 1, wherein the pharmaceutical administration form is for oral use.

12. The process for manufacture of a solid pharmaceutical administration form according to claim 11, wherein the pharmaceutical administration form provides immediate release of the active pharmaceutical ingredient.

13. The process for manufacture of a solid pharmaceutical administration form according to claim 1, wherein the particle size of the mesoporous silica particles is characterized by a d₅₀ value from 1 μm to about 500 μm, preferably from about 1 μm to 100 μm and more preferably from about 10 μm to 50 μm.

14. The process for manufacture of a solid pharmaceutical administration form according to claim 1, wherein the particle size of the mesoporous silica particles is characterized by a d₉₀ value of 500 μm or less, preferably 100 μm or less and more preferably 60 μm or less, for instance between 40 μm and 60 μm.

15. The process for manufacture of a solid pharmaceutical administration form according to claim 1, wherein the mesopores in the mesoporous silica particles have a mean diameter from 2 to 50 nm.

16. The process for manufacture of a solid pharmaceutical administration form according to claim 1, wherein a drying step is performed after step (f).

17. The process for manufacture of a solid pharmaceutical administration form according to claim 16, wherein the drying step is performed in the presence of an entrainer.
